(19) United States
(12) Patent Application Publication
(10) Pub. No.: US 2005/0147689 A1
(43) Pub. Date: Jul. 7, 2005

(54) Method for inhibiting the growth of gastrointestinal tract tumors

(76) Inventor: Nejat K. Egilmez, East Amherst, NY (US)

Correspondence Address: HODGSON RUSS LLP, ONE M & T PLAZA, SUITE 2000, BUFFALO, NY, 14203-2391, US

(21) Appl. No.: 10/748,003

(22) Filed: Dec. 30, 2003

(51) Int. Cl. A61K 38/20; A61K 45/00; A61K 9/16; A61K 9/50

(52) U.S. Cl. 424/490; 424/85.2; 264/4.1

(57) ABSTRACT

The present invention provides compositions and methods for preventing the development of or reducing the growth of gastrointestinal tumors. The composition comprises polymeric microspheres encapsulating sulindac, IL-12 or both. These polymeric microspheres can be administered orally to individuals to reduce the growth of or prevent the development of gastrointestinal tumors.

### FIELD OF THE INVENTION

The present invention relates generally to the field of cancer and more particularly to a method of preventing or reducing the growth of gastrointestinal tumors by oral administration of encapsulated IL-12, sulindac or both.

### DISCUSSION OF RELATED ART

Gastrointestinal (GI) tract malignancies, which include esophageal, gastric, intestinal and colorectal cancers, are only second to lung cancer in cancer-related mortality in the U.S. population. Of these, colorectal cancer is the most common (10% of all cancer deaths), followed by esophageal, gastric and the intestinal cancers (4, 4 and 0.5% of all cancer deaths, respectively). In the year 2003, about 82,000 people are expected to die from GI cancer in the U.S. alone.

Colorectal cancer is the third most prevalent form of cancer and third most frequent cause of cancer-related death in the United States (1, 2). Evidence suggests that colorectal cancer arises from preexisting polyps and necropsy studies have shown a premalignant polyp prevalence of 35% in Europe and USA (1). Both genetic predisposition and environmental factors are recognized as factors contributing to the development of colorectal cancer (1, 2). Two main inherited disposition syndromes are familial adenomatous polyposis (FAP) and hereditary non-polyposis colorectal cancer (HNPCC). These syndromes represent roughly 1% and 6% of all colorectal cases (1). Sporadic colorectal cancer which represents the overwhelming majority of the cases is thought to arise in a multistep accumulation of mutations in tumor suppressor genes and oncogenes (3) and is mainly seen in the elderly. The current colorectal prevention strategies focus on the identification and surgical removal of the premalignant polyps while the treatment for established disease involves surgical resection. Development of non-surgical preventive therapies for individuals at risk and non-surgical adjuvant therapies for the treatment of established and advanced disease would have significant public health benefit.

The development of colorectal cancer is a multistep event that can take 5-10 years as the adenomatous polyps develop from tubular to villous adenomas and finally to malignant tumors (1). It has been shown that dietary and pharmacological intervention can inhibit this process significantly. Particularly promising are a group of agents called the non-steroidal anti-inflammatory drugs (NSAIDs). Sulindac, a potent NSAID which is generally used for the treatment of rheumatic disorders, has been effective in the prevention and treatment of pre-malignant and malignant colonic polyps both in preclinical murine tumor models (4, 5) and in human patients with familial adenomatous polyposis (FAP) (9-11). Repeated administration of sulindac to C57B1/6J-Min (Min) mice suppresses the development of adenomatous polyps and eradicates established tumors (4-6). Moreover sulindac has been shown to inhibit tumor development in chemically induced tumor models in rodents (7, 8). In human clinical trials involving FAP patients, administration of Sulindac resulted in a 40-50% decrease in both the number and the size of the polyps (9-11). In these patients, discontinuation of sulindac administration resulted in the reappearance of the polyps. Sulindac has been shown to inhibit cyclooxygenase which results in reduced levels of prostoglandins. Elevated levels of prostoglandins are linked to the development of colon tumors. A number of other studies suggest a prostoglandin-independent mechanism for the antitumor activity of sulindac as well (6, 12, 13). In all these studies, sulindac was administered orally in a soluble form.

Sulindac is reversibly metabolized within the body to form the active metabolite sulindac sulfide. It is believed that sulindac is converted to sulindac sulfide in the liver, kidneys, and gut (via gut microflora) and that the local concentration of sulindac sulfide within the intestinal lumen is the active agent in the inhibition of tumor growth (14, 15). Yet it is not currently clear whether direct local delivery (through intra-luminal conversion of sulindac into its active metabolite) or the systemic absorption (and subsequent conversion into sulindac sulfide in the liver which is then reabsorbed into the intestines) results in the inhibition of intestinal adenomas.

NSAIDs, when administered in free drug form (tablets), can produce a sticky agglomerate upon coming into contact with gastric juice resulting in a high local concentration, reduced absorption and gastric irritation. Additionally frequently observed adverse clinical reactions with the oral delivery of sulindac include dyspepsia, nausea, vomiting, diarrhea and gastrointestinal cramps, headache, psychic disturbances, vertigo and edema. Other less frequent, but serious, adverse reactions to sulindac tablets include pancreatitis, renal toxicity, and congestive heart failure. There has been heretofore no demonstration of the efficacy of oral sulindac for reducing the growth of intestinal tumors.

While chemotherapeutic drugs represent standard therapy, immunotherapy has recently emerged as a promising new modality for cancer treatment (16, 17). Immunotherapy introduces the possibility of long-term protection from recurrence by promoting the development of systemic antitumor immunity which is not achievable by chemotherapeutic drugs. The potential of various immunotherapeutic approaches, especially that of immunostimulatory cytokines, has been demonstrated in numerous preclinical tumor models (reviewed in 17, 18). While highly effective in murine models, most cytokines display serious toxicity when administered systemically in human patients (19, 20). Recent studies have focused on the development of technologies for the low-level, local and sustained delivery of cytokines directly to the tumor site such as gene-modified cells to avoid systemic toxicity (21, 22). Although gene-modification has worked well in preclinical models, its application in the clinics has been difficult due to the expense and the complicated technology (18, 21). Therefore the development of clinically feasible and less expensive technologies for the local and sustained release of cytokines or drugs at the tumor site are highly desirable.

Of the numerous cytokines tested in murine tumor models, IL-12 has induced the most dramatic regression of established tumors with the concomitant development of systemic antitumor immunity (21-23). Both systemic and local delivery of IL-12 is effective but high-dose systemic delivery necessary to effect tumor growth inhibition is associated with prohibitive toxicity in humans (22). IL-12 gene-modified tumor cell vaccines are currently in clinical trials and some induction of antitumor immunity in patients has been reported (24). The use of immunotherapy to treat gastrointestinal tumors has been limited both in the preclinical models and in human patients since the local and sustained delivery of cytokines to gastrointestinal tumors is difficult (1). Accordingly, there is a need in the field of gastrointestinal cancers for novel therapeutic approaches.

### SUMMARY OF THE INVENTION

The present invention provides compositions and method for reducing the incidence of, or reducing the growth of gastrointestinal tumors. The formulations comprise polymeric microspheres encapsulating sulindac, IL-12 or both. Sulindac encapsulating microsphere formulations were observed to be superior to soluble drug in reducing the incidence of the development of, intestinal tumors in mice. The results also demonstrated that sulindac microspheres of the present invention are highly effective in inducing the regression of established tumors in adult mice. Oral administration of IL-12-encapsulated microspheres was observed to promote the suppression of established tumors. In addition, combined treatment with IL-12- and sulindac-encapsulated microspheres was found to be superior to either formulation alone in inducing tumor regression.

In one embodiment, the polymers for preparing the microspheres include polyanhidrides. These include polylactic acid (PLA), polylactide-co-glycolide (PLGA), polycaprolactone (PCL) and poly(fumaric-co-sebacic anhydride) (p(FA:SA). Accordingly, in one embodiment of the invention, a method is provided for reducing the growth of gastrointestinal tumors by oral administration of a drug composition comprising sulindac encapsulated in polymeric microspheres.

In another embodiment the drug formulation encapsulated in the polymeric microspheres comprises IL-12. In yet another embodiment, the drug formulation comprises both sulindac and IL-12. For administration of both IL-12 and sulindac, microspheres can be loaded with both IL-12 and sulindac or separate microspheres loaded with IL-12 or sulindac can be used.

In another embodiment, the polymeric microspheres encapsulating sulindac, IL-12 or both can be used to reduce the incidence of gastrointestinal tumors. In accordance with this embodiment, the polymeric microspheres loaded with sulindac, IL-12 or both are used to partially or fully prevent gastrointestinal tumors.

### BRIEF DESCRIPTION OF THE DRAWINGS

FIG. 1 is a representation of the release profile of 10% poly(fumaric-co-sebacic anhydride) microspheres encapsulating sulindac prepared by the hot melt method. The amount of release of sulindac is shown as a function of time.

FIG. 2 is a representation of the release profile of 5% polylactic acid microspheres encapsulating sulindac prepared by the phase inversion method. The amount of released sulindac is shown as a function of time.

FIG. 3 is a representation of the release profile from four different batches of 5% poly(fumaric-co-sebacic anhydride) microspheres encapsulating sulindac prepared by the phase inversion method. The amount of released sulindac is shown as a function of time.

FIG. 4A is a representation of the 7 day release profile from polylactic acid microspheres encapsulating IL-12 prepared by the phase inversion method.

FIG. 4B is a representation of the bioactivity of the released IL-12 showing specific activity in the IL-12 released after 1 day as a percentage of unencapsulated IL-12.

FIG. 5 is representation-of the effect of administration of encapsulated sulindac according to the present invention on the development of tumors. Data are shown for soluble sulindac and polymeric microspheres encapsulating sulindac prepared by the hot-melt or phase inversion methods.

FIG. 6 is a representation of the effect of administration of PBS or blank microspheres—either polylactic acid microspheres prepared by the phase inversion method or poly(fumaric acid co-sebacic acid) prepared by the hot melt method on the development of tumors.

FIG. 7 is a representation of the effect of polylactic acid microspheres prepared by the phase inversion method (PLA-PIN) or poly(fumaric acid co-sebacic acid) microspheres prepared by the phase invertion (pFA:SA-PIN) or the hot melt methods (pFA:SA-HM) encapsulating sulindac on the development of intestinal tumors.

FIG. 8 is a representation of the effect of polylactic acid microspheres made by the phase invertion (PLA-PIN) and encapsulating sulindac on the development of intestinal tumors. The administration dosage was 3, 8 and 20 mg corresponding to approximately 0.3, 0.8 and 2 mg of sulindac per feeding.

FIG. 9 is a representation of the effect of the frequency of administration of the polymeric microspheres of the present invention on the development of intestinal tumors. 0.8 mg of drug equivalent for sulindac was given in polylactic acid microspheres prepared by the phase inversion method two or five times a week.

FIG. 10A is a representation of the effect of sulindac loaded polylactic acid microspheres prepared by phase inversion method to induce regression of established tumors.

FIG. 10B is a representation of the effect of sulindac loaded polylactic acid microspheres prepared by the hot-melt method to induce regression of established tumors.

FIG. 11 is a representation of the effect of administration of sulindac loaded, IL-12 loaded, or co-administration of sulindac loaded and IL-12 loaded microspheres on the regression of established tumors. Control mice received saline.

### DETAILED DESCRIPTION OF THE INVENTION

The present invention provides compositions and methods for inhibiting the growth of or preventing the development of intestinal tumors by oral administration of sulindac, IL-12 or both in an encapsulated oral formulation. The formulation of the present invention comprises encapsulating the sulindac or IL-12 or both in polymer microspheres. The formulation can be used for both reducing the incidence of, or reducing the growth of intestinal tumors.

The term “gastrointestinal tumor” as used herein means any tumors of the gastrointestinal tract which includes esophagus, stomach and the small and large intestines.

The polymeric microspheres of the present invention comprise polymers including hydrophilic polymers such as those containing carboxylic groups, such as poly(acrylic acid). Rapidly bioerodible polymers such as poly(lactide-co-glycolide), polyanhydrides, and polyorthoesters having carboxylic groups exposed on the external surface as their smooth surface erodes, are particularly useful. Other representative synthetic polymers include polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, celluloses including alkyl cellulose, hydroxyalkyl celluloses, cellulose ethers, cellulose esters, and nitrocelluloses, polymers of acrylic and methacrylic esters, poly(lactide-co-glycolide), polyanhydrides, polyorthoesters blends and copolymers thereof.

In general, the polymeric microspheres are slow-release biodegradable particles. The particles should have adequate uptake in the GI tract and be such that the release rate provides for sufficient release of the drug. In one embodiment, the polymeric biospheres are bioadhesive which is considered to increase the transit time of the particles in the GI tract. In one embodiment, thermoplastic polyanhidride polymers are used. These include polylactic acid (PLA), polylactide-co-glycolide (PLGA), polycaprolactone (PCL) and poly(fumaric-co-sebacic anhydride) (p(FA:SA).

In one embodiment described herein, the microspheres contain blends of two or more biodegradable polymers, preferably poly(hydroxy acids) of different molecular weight and/or monomer ratio. For example, different molecular weight polymers can be blended to form a composition that has linear release over a defined period of time, ranging from at least one day to several days. Thus, the release window can be varied by adjusting the molecular weight of the polymers used.

While not intending to be bound by any particular theory, it is considered that bioadhesive microspheres improve absorption by prolonging the intestinal passage time of the drug and extend pharmacokinetic half-life by the slow, sustained release of the drug (particularly by the PIN microspheres). Administration of the drug via dispersed slow-release vehicles may also reduce adverse side effects.

The polymeric microspheres can be prepared by well known technologies (see Mathiowitz et al. Controlled Release 5, 13-22 (1987); Mathiowitz, et al., Reactive Polymers 6, 275-283 (1987); and Mathiowitz, et al., J. Appl. Polymer Sci. 35, 755-774 (1988), U.S. Pat. No. 6,235,313). The selection of the method depends on the polymer selection, the size, external morphology, and crystallinity that is desired, as described, for example, by Mathiowitz, et al., Scanning Microscopy 4, 329-340 (1990); Mathiowitz, et al., J. Appl. Polymer Sci. 45, 125-134 (1992); and Benita, et al., J. Pharmn. Sci. 73, 1721-1724 (1984). These methods include solvent evaporation, phase separation, spray drying, and hot melt encapsulation.

In the PIN method, nano-seized microspheres are fabricated by the spontaneous phase inversion of dilute polymer solutions that are quickly dispersed into an excess of non-solvent for the polymer. This method differs from existing methods of encapsulation in that no stirring or agitation of the non-solvent bath is required. Moreover there are no aqueous phases involved in the process which provides for high encapsulation efficiencies for hydrophillic molecules.

In the hot melt method the polymer is first melted and then mixed with the solid particles of the drug that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decantation with petroleum ether to give a free-flowing, powder. Microspheres with sizes between one to 1000 microns are obtained with this method. The external surfaces of spheres prepared with this technique are usually smooth and dense. This procedure is used to prepare microspheres made of polyesters and polyanhydrides. However, this method is not preferred for preparing microspheres encapsulating IL-12 since the temperature required during the preparation process is likely to denature IL-12 so as to render it inactive.

The oral formulation of the present invention may contain microspheres loaded with IL-12, sulindac and/or both. Further, the microspheres encapsulating IL-12 or sulindac may be made from different polymers and by different method. For example, the sulindac microspheres may be prepared by the PIN or the HM methods while it is preferable to prepare the IL-12 microspheres by the PIN methods. Accordingly, if microspheres encapsulting both IL-12 and sulindac are to be prepared, the preferred method of preparation is the PIN method.

The microspheres of the present invention can be administered in suspension. Pharmaceutically acceptable carriers for oral administration are known and determined based on compatibility with the polymeric material. The dosage and administration of sulindac and IL-12 are well within the purview of those skilled in the art. For example, the dosage of sulindac in humans can be about 100-400 mg/patient. The dose can be given as needed (such as twice a day). The treatment can be continued as needed (for example, for 3-6 months). For IL-12, the dose may be 100-300 ng/kg per patient, which can be administered, for example for 5 days every 3 weeks for 34 cycles. Both sulindac and IL-12 are used clinically and therefore the dosage and administration regimens are well known. Those skilled in the art will recognize that the dosage for the present invention would be typically less than what is used for the soluble form since the encapsulated drugs are being delivered locally and in a slow release form.

The polymeric microspheres of the present invention are useful for preventing tumors of the GI tract. In accordance with this embodiment, the polymeric microspheres encapsulating sulindac, IL-12 or both may be administered orally to individuals in combination with other forms of treatment such as surgery, radiation, chemotherapy or immunotherapy. Thus, the polymeric microspheres may be administered prior to, during and/or after surgery, radiation, chemotherapy or immunotherapy. In addition, the polymeric microspheres may also be administered to individuals who are considered to be at risk of developing intestinal tumors. Such risk assessment may be based on several factors known to those skilled in the art including environmental factors, heredity, diet and the like.

The polymeric microspheres of the present invention are also useful for inhibiting the growth of existing tumors. It should be understood that by the term “inhibiting” is meant a reduction in the growth over expected growth in the absence of encapsulated sulindac or IL-12. In accordance with this embodiment, the polymeric microspheres encapsulating sulindac, IL-12 or both can be orally administered to individuals having one or more gastrointestinal tumors.

The following examples will further describe the present invention. It should be noted that these examples are illustrative and are not intended to be restrictive in any way.

### EXAMPLE 1

This embodiment describes the preparation of pFA:SA microspheres by the HM method. Monomers of fumaric acid and sebacic acid were obtained from a commercial source (Aldrich Chemicals) and the polymer of polyfumaric-co-sebacic anhydride, prepared in accordance with established protocols for the hot melt method (see Mathiowitz et al.), were prepared by Sperics, Inc., Lincoln, R.I. Polymeric-co-sebacic anhydride microspheres were prepared by heating the polymer to about 10° C. above its melting point and sulindac was added to the melted polymer slowly. The slurry was stirred vigorously and the melted polymer with drug was poured into a stirred hot silicon oil bath which was 10° C. above the melting point of the polymer (about 80° C.). The oil bath contained 4 drops of a surfactant (Span 85). An overhead impeller was used to stir the oil/spheres. Once the polymer and drug combination had been poured into the hot oil bath, an ice-water bath was placed around the oil bath for rapid cooling and the stirring continued until the oil bath was at room temperature. The spheres were collected via filtration and washed with petroleum ether to remove the oil. The final composition of the microsphere formulations was about 5-20% sulindac and 95-80% polymer.

### EXAMPLE 2

This example describes the preparation of microspheres by the PIN method described in detail previously (see Mathiowitz et al., 1997, Nature, 386:410-414). Briefly, bovine serum albumin (BSA) the polyfumaric acid, polysebacic acid and sulindac in methylene chloride were rapidly poured into light petroleum for formation of microspheres. IL-12 can be added in methylene chloride (see 26, 30). Microspheres were filtered and lyophilized overnight for complete removal of solvent and stored at 4° C. The final composition of the microspheres was about 1% BSA, 1% IL-12, and 98% polymer or 5-20% sulindac and 80-90% polymer.

### EXAMPLE 3

This embodiment describes the fabrication and characterization of sulindac and IL-12-loaded microspheres. Three different formulations of sulindac-encapsulated microspheres were prepared using the hot-melt (HM) and phase inversion nanoencapsulation (PIN) technologies. Poly-fumaric-co-sebacic anhydride (pFA:SA) was used for formation of HM microspheres whereas either polylactic acid (PLA) or pFA:SA were utilized for PIN formulations. The formulations were then characterized for particle size, loading and release kinetics.

a) Loading and size. Loading of sulindac was 10% (w/w) for HM and either 5% or 10% for PIN microspheres. The HM microspheres were sieved to a size range of 25-212 μm. The PIN spheres are typically smaller than the HM spheres. The PIN spheres for the present invention are preferably in the range of 0.1 to 10 μm. The PIN spheres from different batches of formulation preparations were sized using a Coulter Particle Size analyzer, and typical size for PIN microspheres is shown in Table 1:

TABLE 1

| | Size Distribution - based | | Size Distribution - based | |
|---|---|---|---|---|
| | on Number (μm) | | on Volume (μm) | |
| Formulation | <50% | 90% | <50% | <90% |
| CS00021.27A | 1.172 | 1.440 | 1.474 | 9.791 |
| CS00021.27B | 1.253 | 1.629 | 1.924 | 10.63 |
| CS00021.27C | 1.206 | 1.484 | 1.480 | 8.418 |
| CS00021.27D | 1.200 | 1.538 | 1.663 | 10.09 |
| CS00021.27E | 1.208 | 1.555 | 1.887 | 11.22 |
| CS00021.27F | 1.237 | 1.837 | 2.013 | 6.572 |
| CS00021.27G | 1.192 | 1.560 | 2.088 | 11.20 |
| CS00021.27H | 1.179 | 1.434 | 1.367 | 8.400 |

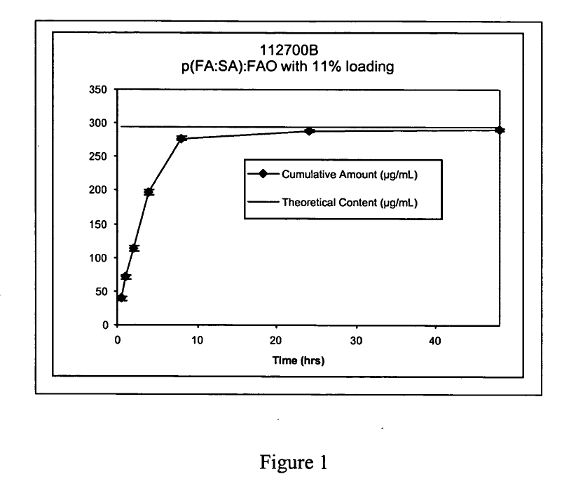

b) Release kinetics. Three different formulations were evaluated for release kinetics. Approximately 90% of sulindac was released from the PLA-PIN microspheres within 48 hours. The release of drug was faster from the larger HM microspheres, due in large part to the type of polymer used. p(FA:SA) degrades much faster than PLA, and ˜90% of drug was released within the first 9 hours from the hot melt microspheres. In the case of the pFA:SA-PIN microspheres, the release was even more dramatic in that 85-90% of encapsulated drug was released within 2 hours. Release profiles are shown in FIGS. 1-3.

FIG. 1 shows release profile of sulindac from 10% pFA:SA hot melt microspheres. Release was performed by incubating 40 mg of microspheres in 15 mL PBS, pH 7, 37° C. At each time point, 1 mL of release buffer was removed and analyzed for drug content via HPLC. An equivalent amount of buffer was replaced. Release was performed in triplicate.

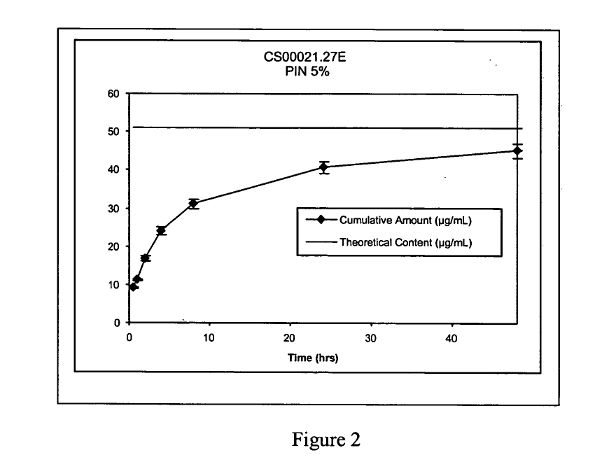

FIG. 2 shows release profile of sulindac from 5% PLA PIN microspheres. Release was performed by incubating 10 mg of microspheres in 10 ml PBS, pH 7, 37° C. At each time point, 1 ml of release buffer was removed and analyzed for drug content via HPLC. An equivalent amount of buffer was replaced. Release was performed in triplicate. The release profile of sulindac from PLA-PIN microspheres with 10% loading was similar (data not shown).

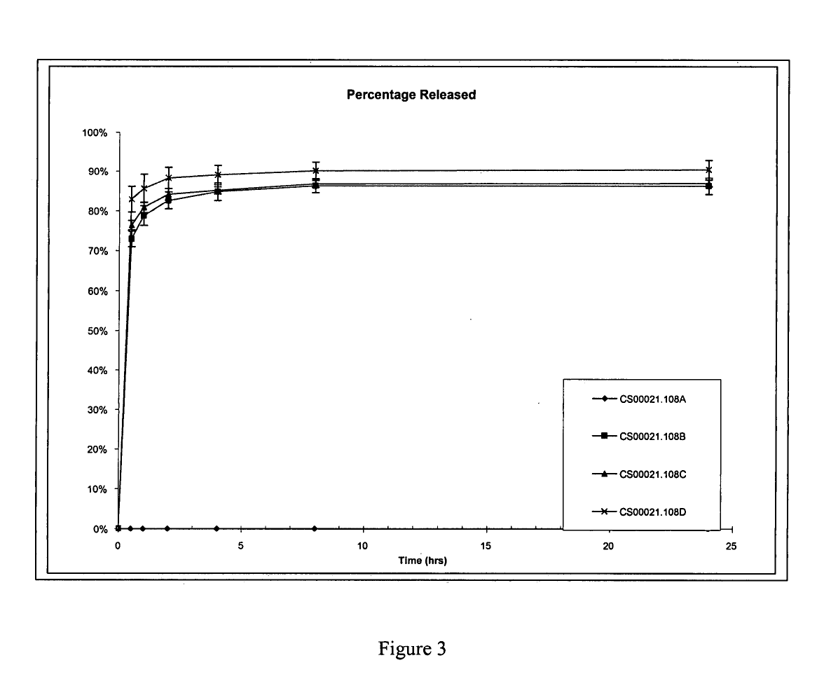

FIG. 3 shows release profile of sulindac from 5% pFA:SA PIN microspheres. Release was performed by incubating 10 mg of microspheres in 10 ml PBS, pH 7, 37° C. as described above (FIG. 2). Release profiles of 4 different batches are shown (triplicate samples). The release profiles of sulindac from 10% and 5% pFA:SA-PIN microspheres were essentially identical.

### EXAMPLE 4

This example describes the preparation of IL-12 loaded microspheres. These microspheres were prepared using the PIN technology alone since the hot-melt protocol involves heating of the polymer to 90° C., which denatures and inactivates the protein. Polylactic acid was used as the polymer of choice since poly-fumaric-sebacic acid formulation reduced the bioactivity of the cytokine (data not shown). The encapsulation of IL-12 into PLA-PIN particles was done as previously described (1). Briefly, bovine serum albumin (BSA, RIA grade, Sigma Chemical Co., St. Louis, Mo.), polylactic acid (PLA, MW 24,000 and MW 2,000 [1:1, wt/wt], Birmingham Polymers, Inc, Birmingham, Ala.), and recombinant IL-12 in methylene chloride (Fisher, Pittsburgh, Pa.) were rapidly poured into petroleum ether (Fisher, Pittsburgh, Pa.) for formation of microspheres. Microspheres were filtered and lyophilized overnight for complete removal of solvent. The final formulation contained 1% BSA (wt/wt) and 1% murine IL-12 (˜10 μg [270,000 U]/mg PLA).

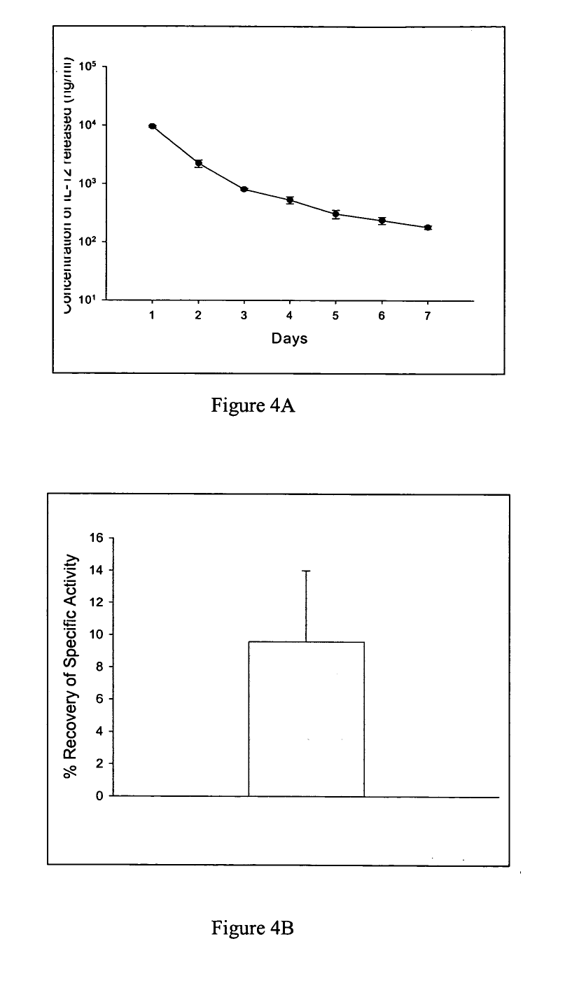

The release profile and the bioactivity of IL-12 that was released from the microspheres were determined using previously described in vitro assays (25). The 7-day release profile and bioactivity of murine recombinant IL-12 from PLA PIN microspheres were carried out. Briefly, 2 mg of microspheres were suspended in 200 μl of complete culture medium (DMEM-F12+10% FCS) and incubated at 37° C. in a CO₂ incubator. Supernatant was collected daily and the IL-12 concentration was determined using a murine p70 IL-12-specific ELISA (Pierce-Endogen). The results are shown in FIG. 4A. Bioactivity of the released IL-12 was determined using an activated T-cell proliferation assay as described by us (26). Specific activity of day 1 samples (in triplicate) were determined and compared to that of an equal amount of unencapsulated IL-12. The percent recovery of specific activity after encapsulation and release from the microspheres is shown in FIG. 4B.

The results shown in FIG. 4 establish that IL-12 is released from the PLA-PIN microspheres at physiologically relevant quantities for at least 7 days. Moreover the IL-12 that is released is still bioactive although the specific activity is approximately 10% that of unencapsulated protein.

### EXAMPLE 5

This example describes the ability of sulindac-loaded microspheres prepared by HM or PIN to prevent the development of intestinal adenomas. The formulations were tested in vivo, in young tumor-free APC/Min^{+/-} mice, to determine whether the local and sustained release of sulindac from the microspheres to the gastrointestinal tract was superior to free drug in preventing tumor development.

Heterozygous C57B1/6J-Min (Min/+) mice spontaneously develop multiple intestinal adenomas due to a germ-line mutation in one allele of the murine homolog of the human APC gene (27, 28). The Min mouse is a model for human intestinal cancers with similarities to an inherited form of human intestinal cancer, familial adenomatous polyposis (FAP). The homozygous Min/Min mice are not viable whereas 100% of the heterozygous animals develop intestinal adenomas. These mice develop 30-50 intestinal tumors by 20 weeks of age and die from tumor-associated anemia or intestinal obstruction by 21-22 weeks of age (27). The adenomas that develop contain multiple epithelial lineages, suggesting that the defect arises in a pluripotent epithelial stem cell. Also, these adenomas show a loss of the wild type APC locus, consistent with the tumor suppressive role of the APC gene. Unlike tumors in humans with FAP, which arise in the large intestine and duodenum, tumors in Min mice are primarily found in the small intestine. The Min mouse has been extensively used to evaluate chemopreventive agents, primarily sulindac.

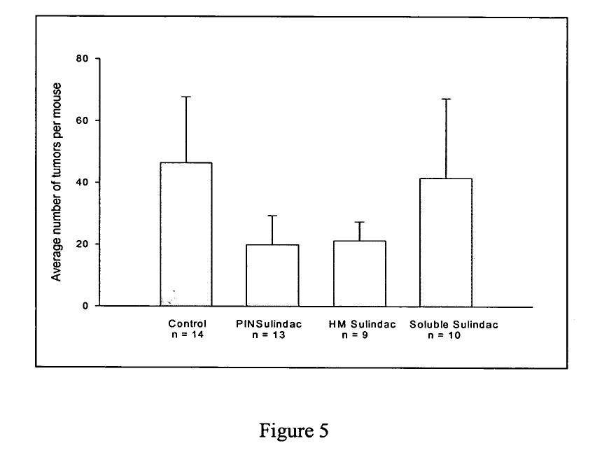

Drug in encapsulated form (HM-sulindac and PIN-sulindac) was compared to free drug and saline controls. The results from these studies are shown below in FIG. 5. Mice were fed orally with 0.3 mg of sulindac per feeding using the following formulations. PBS alone (no sulindac), sulindac-encapsulated HM microspheres (10% loading), sulindac-encapsulated PIN microspheres (10% loading), or free sulindac. All formulations were in 0.2 ml PBS+15% ethanol per feeding. The feedings started when the mice were 6 weeks of age. Mice were fed twice a week for 6 weeks. They were then sacrificed at 12 weeks of age, intestines were removed, flushed with Kreb's Ringer's solution, fixed overnight in 10% formalin and were analyzed for tumors using a dissecting microscope. The number of mice in each group is shown in the graph (n). The differences between the control (PBS alone) versus PIN-Sulindac and HM-Sulindac groups were highly significant (p≦0.003). The differences between soluble sulindac group versus PIN-Sulindac and HM-Sulindac were also significant (p=0.010 and 0.033, respectively). There was no difference between the control and the soluble sulindac groups (p=0.72). These data are a combination of two separate experiments.

The results shown in FIG. 5 establish that both sulindac-encapsulated HM and PIN microspheres are superior to soluble sulindac in suppressing the development of tumors in young mice, with the PIN formulation having a slight advantage over the HM formulation (p=0.01 and 0.033 for PIN vs soluble sulindac and HM vs. soluble sulindac, respectively). At the given dose and schedule, free sulindac had no effect on tumor growth (compared to control group, p=0.72).

### EXAMPLE 6

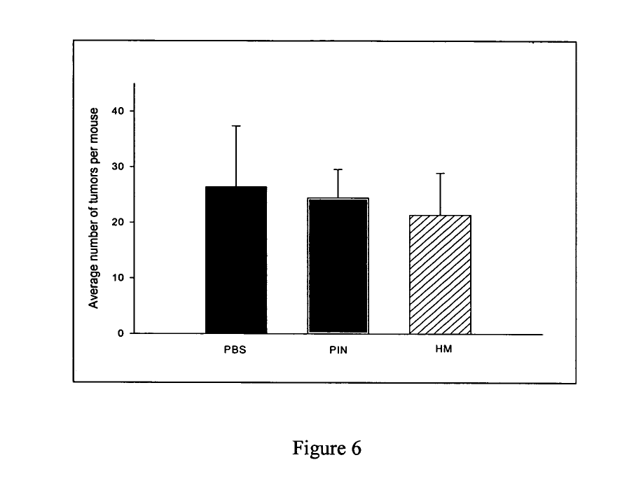

The question of whether the microspheres themselves were having a non-specific effect on tumor development was addressed in this experiment. Six-week old APC/Min^{+/-} mice were divided into 3 groups and were fed PBS, blank PIN or blank HM microspheres. The treatments were twice a week for 6 weeks (3 mg of polymer per feeding (0.3 mg sulindac). The mice were then sacrificed and their intestines analyzed for tumor nodules. The results are shown in FIG. 6.

Six-week old mice were fed either with PBS, PLA PIN or pFA:SA HM microspheres (3 mg) twice a week for 6 weeks. They were then sacrificed and the intestinal tumors were quantified. There was no difference between PBS and microsphere groups (p=0.64 and 0.29 for PIN and HM, respectively). Bars=standard deviation, n=7 for PBS and 9 for microsphere groups. These results indicate that blank microspheres do not suppress intestinal tumor development in the young Min^{+/-} mice.

### EXAMPLE 7

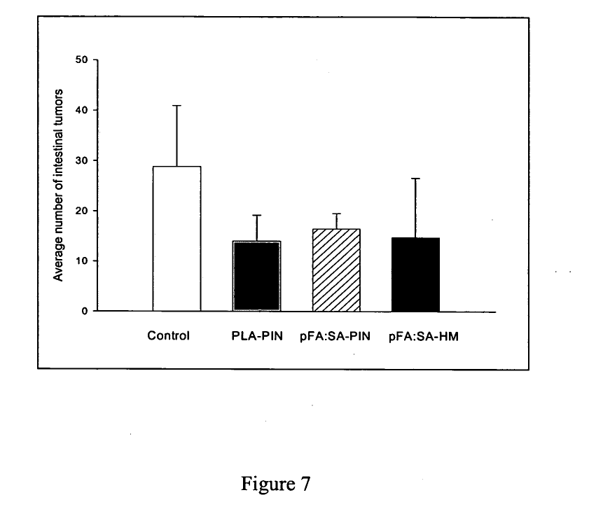

This example describes a comparison of different polymer formulations. In initial experiments, PLA-PIN formulation was compared to pFA:SA HM formulation and both formulations displayed similar activity. Both size (superior uptake in the intestinal mucosa for smaller particles) and bioadhesive characteristics (longer passage time) can influence the therapeutic efficacy of slow-release particles in the GI tract. We expected that while the small size of the PLA-PIN particles (<2 micron) was important to their efficacy, the bioadhesive properties of the large pFA:SA HM microspheres would be their primary advantage. A third alternative was to produce small, bioadhesive particles (pFA:SA-PIN) to improve efficacy. To this end pFA:SA-PIN particles were tested against the earlier formulations. Young, tumor-free mice were fed either with the PLA-PIN, pFA:SA-PIN or pFA:SA-HM microspheres loaded with sulindac. The results are shown in FIG. 7.

Young APC/Min^{+/-} mice (6-weeks old) were fed with the sulindac-encapsulated formulations (0.3 mg sulindac, 10% loading for all) twice a week for 6 weeks. Control group received PBS alone. The differences between the control and the PIN formulations were significant (p=0.015 and 0.025 for PLA-PIN and pFA:SA-PIN, respectively). The difference between the control and the pFA:SA-HM groups was not statistically significant although the average number of intestinal tumors was lower in pFA:SA-HM than in control. Bars=standard deviation (n=5, 7, 7 and 6 for the control, PLA-PIN, pFA:SA-PIN and pFA:SA-HM groups, respectively). PIN formulations were found to be generally more effective than the HM formulations in preventing intestinal tumor development in young APC/Min^{+/-} mice.

The results shown in FIG. 7 establish that the pFA:SA-PIN formulation work as well as the PLA-PIN formulation. It is possible that while the bioadhesive properties of pFA:SA formulation increases the passage time of the spheres, the rapid degradation and release of drug from the pFA:SA (see FIG. 3) cancels this effect out.

### EXAMPLE 8

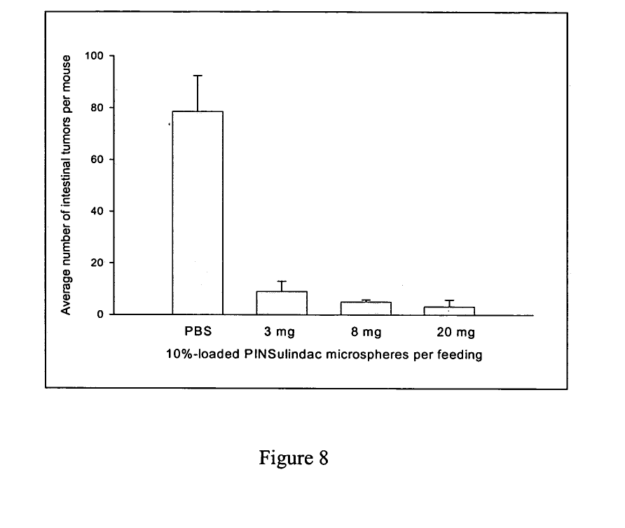

This embodiment demonstrates the effect of drug dose and treatment frequency on tumor suppression. The effect of increasing the drug dose on the efficacy of tumor suppression was evaluated in the next series of experiments. The PLA-PIN formulation was used in these studies. The results are shown in FIG. 8.

Young mice (5.5 weeks old) were fed with increasing amounts of PLA-PIN sulindac (10% loading) microspheres (3, 8 and 20 mg microspheres corresponding to 0.3, 0.8 and 2 mg of sulindac per feeding) twice a week for 6 weeks. Control mice received saline. Mice were sacrificed two days after the last feeding and the intestines were analyzed for tumor burden. Bars=standard deviation, n=5 per group.

The results shown in FIG. 8 establish that increasing the dose of sulindac enhanced tumor suppression significantly. The average number of tumors per mouse declined from 79 in the control group to 9, 5 and 3 in the 0.3, 0.8 and 2 mg drug groups, respectively. The differences between the control and the treatment groups were highly significant (p≦0.000005). The differences between the group that received 0.3 mg sulindac per feeding, and the 0.8 mg and 2 mg groups were also significant (p=0.053 and 0.026, respectively). The difference between the 0.8 mg and 2 mg groups was not significant (p=0.22).

### EXAMPLE 9

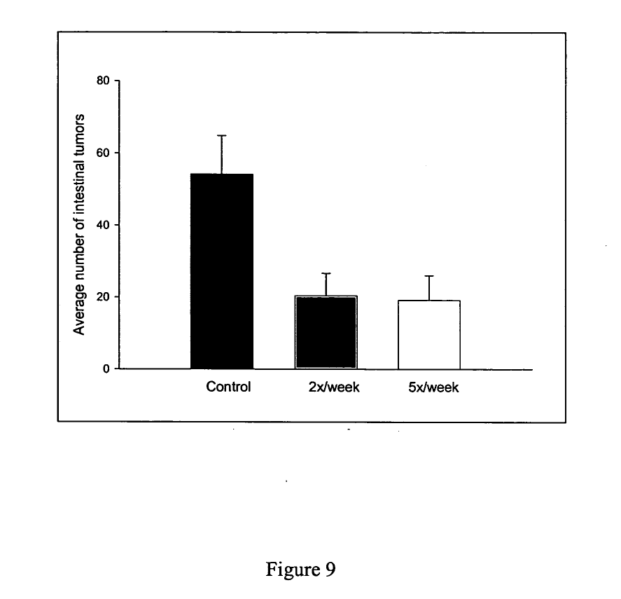

In this example we tested the effect of treatment frequency on tumor development in young mice. Young mice (5.5 weeks old) were fed orally with PLA-PIN sulindac microspheres (10% loading, 0.3 mg drug per feeding) either twice or 5 times a week for 6 weeks. Control mice were fed saline twice a week. They were then sacrificed and the intestines were analyzed for tumor load. The results are shown in FIG. 9. Bars=standard deviation, n=6, 7, and 7 for control, 2× per week and 5× per week, respectively.

The data shown in FIG. 9 demonstrate that increasing the frequency of treatment does not enhance the efficacy of tumor suppression. The average numbers of tumors per group were 54, 20 and 19 for control, 2×/week and 5×/week groups respectively. The differences between treatment and control groups were highly significant (p≦0.00002), however there was no difference between the two treatment groups (p=0.72).

The studies described above established that slow-release microsphere formulations are superior to soluble drug in preventing the development of intestinal tumors in the APC/Min^{+/-} model (FIG. 5). Furthermore, the PIN formulation appears to be more consistent than the HM formulation in achieving tumor suppression (FIG. 7). Optimization studies established that with the 10% PLA-PIN Sulindac formulation, the most effective dose was ˜0.8 mg drug equivalents per feeding (FIG. 8). Increasing the frequency of feedings from 2 to 5 times a week did not enhance the anti-tumor activity (FIG. 9).

### EXAMPLE 10

This example demonstrates the ability of sulindac-loaded microspheres prepared by HM or PIN methods to induce the regression of established intestinal adenomas in APC/Min^{+/-} mice. The above results established that encapsulated sulindac could prevent the development of tumors in young mice and were superior to soluble drug in achieving tumor suppression. A more clinically relevant question is whether this approach would be effective in achieving the regression of established intestinal tumors. To this end, the efficacy of sulindac-loaded microspheres in inducing the regression of established tumors was tested in adult mice with advanced disease. Mice were maintained until they were 9 weeks old to allow for tumor development (29). Tumor-bearing mice were then treated with sulindac-loaded PLA-PIN or HM microspheres. Mice were fed 0.8 mg of sulindac (10% loaded PIN or HM microspheres, 8 mg polymer per feeding) 3 times a week for 3 weeks. Mice were sacrificed 3 weeks after the initiation of treatment and the intestines were analyzed for tumor load.

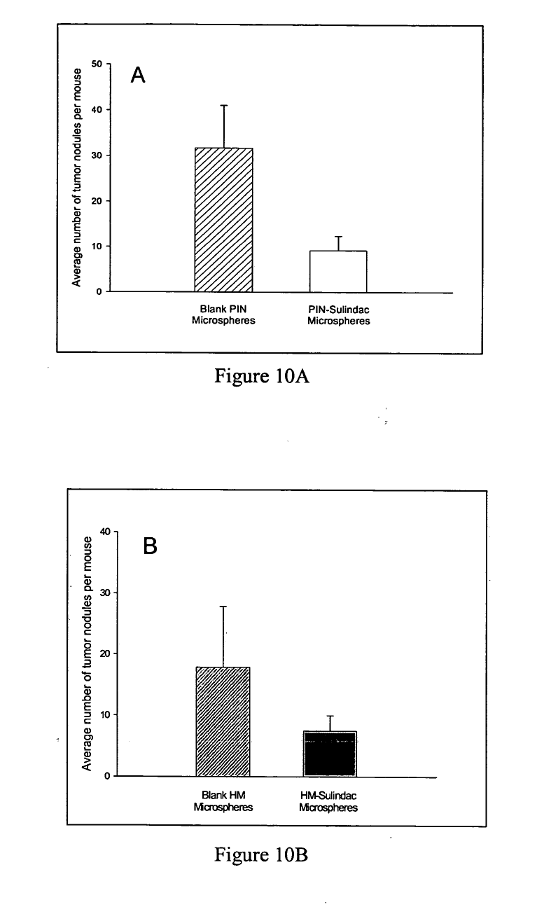

The results are shown in FIG. 10. PIN-Sulindac microspheres induced a highly significant regression of established tumors compared to control blank PIN microspheres (p=0.00009). HM-sulindac microspheres were also effective as compared to blank HM microspheres but the difference was less significant (p=0.021). Bars=standard deviation, n=6, 6, 7 and 7 for blank PIN, blank HM, PIN-sulindac and HM-sulindac, respectively.

The results shown in FIGS. 10A and 10B establish that oral administration of sulindac-encapsulated microspheres can induce the regression of established adenomas in adult APC/Min^{+/-} mice in both HM microspheres and PIN microspheres. In this experiment, PIN formulation appeared to be more effective than the HM formulation in achieving this effect.

### EXAMPLE 11

This embodiment demonstrates the effect of co-administration of sulindac and interleukin-12-loaded microspheres in the inhibition and long-term suppression of established intestinal adenomas in the Min^{+/-} mice.

Adult (9 week-old) with established intestinal adenomas mice were fed either PLA-PIN Sulindac- (10% loading, 0.8 mg drug/feeding), PLA-PIN IL-12-(0.03% loading, 2.5 microgram IL-12/feeding) or a combination of the two, 3-times a week for 3 weeks. Control mice received saline. Mice were then sacrificed and intestinal tumor load was determined. Bars=standard deviation, n=10/group.

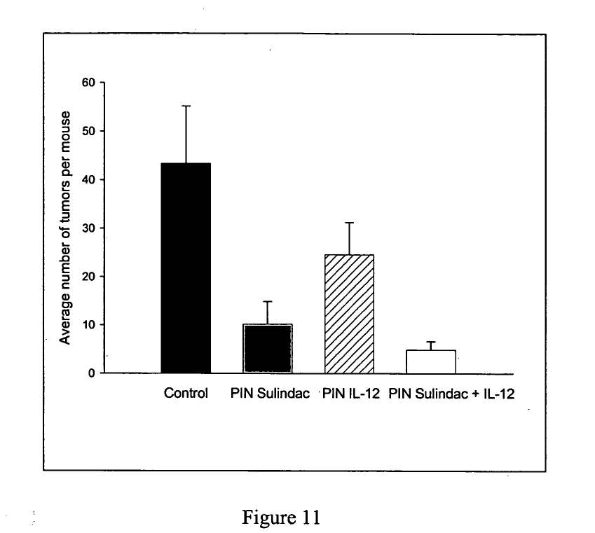

The data shown in FIG. 11 confirms that oral administration of PLA-PIN sulindac microspheres promotes the regression of established intestinal adenomas in adult APC/Min^{+/-} mice (control vs. PIN sulindac, p=1×10⁻⁷). More importantly, these data also establish that oral delivery of PLA-PIN IL-12 microspheres results in a significant reduction in the number of pre-existing adenomas in adult mice (control vs. IL-12 microspheres, p=0.00034). Finally, combined administration of sulindac- and IL-12-encapsulated PLA-PIN microspheres shows a synergistic effect in inducing the regression of established tumors (IL-12+sulindac vs IL-12 alone or sulindac alone, p≦0.003). In limited studies the serum levels of IL-12 and IFNμ (which is produced by IL-12-activated T- and NK cells) were tested to determine whether the IL-1 2 that is released from the microspheres was inducing a systemic immune response. Neither cytokine could be detected in the sera of the treated mice by standard ELISA assays (data not shown) indicating that the IL-12 that is released from the microspheres is most likely acting locally within the gastrointestinal tract.

While the invention has been described through illustrative embodiment, routine modifications to the invention apparent to those skilled in the art are intended to be within the scope of the invention.

References

1. Midgley, R. And Kerr, D. Colorectal cancer. The Lancet 353:391-399, 1999.
2. Garay, C. A. and Engstrom, P. F. Chemoprevention of colorectal cancer: Dietary and pharmacological approaches. Oncology, 13 (1):89-97, 1999.
3. Jen, J., Powell, S. M., Papadopoulos, P., Smith, K. J., Hamilton, S. R., Vogelstein, B. And Kinzler, K. W. Molecular determinants of dysplasia in colorectal lesions. Cancer Res., 54:5523-5526, 1994.
4. Boolbol, S. K., Dannenberg, A. J., Chadbum, A., Martucci, C., Guo, X., Ramonetti, J. T., Abreu-Goris, M., Newmark, H. L., Lipkin, M. L., DeCosse, J. J. and Bertagnolli, M. M. Cyclooxygenase-2 overexpression and tumor formation are blocked by sulindac in a murine model of familial adenomatous polyposis. Cancer Res. 56:2556-2560, 1996.
5. Beazer-Barclay, Y., Levy, D. B., Moser, A. R., Dove, W. F., Hamilton, S. R., Vogelstein, B. And Kinzler, K. W. Sulindac suppresses tumorigenesisin the Min mouse. Carcinogenesis. 17(8):1757-1760, 1996.
6. Chiu, C., McEntee, M. F. and Whelan, J. Sulindac causes rapid regression of preexisting tumors in Min/+ mice independent of prostoglandin biosynthesis. Cancer Res. 57:4267-4273, 1997.
7. Moorghen, M., Ince, P., Finley, K. J. et al. A protective effect of sulindac against chemically induced primary colonic tumors in mice. J. Pathol. 156:341-347, 1988.
8. Piazza, G. A., Alberts, D. S., Hixson, L. J., Paranka, N. S., Li, H., Finn, T., Bogert, C., Guillen, J. M, Brendel, K., Gross, P. H., Sperl, G., Ritchie, J., Burt, R. W., Ellsworth, L., Ahnen, D. J. and Pamukcu, R. Sulindac sulfone inhibits azoxymethane-induced colon carcinogenesis in rats without reducing prostoglandin levels. Cancer Res. 57(14):2609-2915, 1997.
9. Wadell, W. R., Ganser, G. F., Lerise, E. J. et al Sulindac for polyposis of the colon. Am. J. Surg. 57:175-179, 1989.
10. Labayle, D., Fischer, D., Vielh, P. Et al Sulindac causes regression of rectal polyps in familial adenomatous polyposis. Gastroenterology 101:635-639, 1991.
11. Giardello, F. M., Hamilton, S. R., Krush, A. J., et al Treatment of colonic and rectal adenomas with sulindac in familial adenomatous polyposis. N. Engl. J. Med. 328:1313-1316, 1993.
12. Skopinska-Rozewska, E., Piazza, G. A., Sommer, E., Pamukcu, R., Barcz, E., Filewska, M., Kupis, W., Caban, R., Rudzinski, P., Bogdan, J., Mlekodaj, S. And Sikorska, E. Inhibition of angiogenesis by sulindac and its sulfone metabolite (FGN-1): a potential mechanism for their antineoplastic properties. Int. J. Tissue React. 20(3):85-89, 1998.
13. Castonguay, A. and Rioux, N. Inhibition of lung tumourigenesis by sulindac: comparison of two experimental protocols. Carcinogenesis 18(3):491-496, 1997.
14. Mahmoud, N. N., Boolbol, S. K., Dannenberg, A. J., Mestre, J. R., Bilinski, R. T., Martucci, C., Newmark, H. L., Chadburn, A. and Bertagnolli, M. M. The sulfide metabolite of sulindac prevents tumors and restores enterocyte apoptosis in a murine model of familial adenomatous polyposis. Carcinogenesis 19(1):87-91, 1998.
15. Davies, N. M. and Watson, M. S. Clinical pharmacokinetics of sulindac. A dynamic old drug. Clinical Pharmacokin. 32(6):437-459, 1997.
16. Sogn, J. A. Tumor Immunology: The glass is half full. Immunity 9:757-763, 1998.
17 Minev, B. R., Chavez, F. L. and Mitchell, M. S. Cancer Vaccines: Novel approaches and new promise. Pharmacol. Ther. 81(2):121-139, 1999.
18. Colombo, M. P. and Forni, G. Immunotherapy I: Cytokine gene transfer strategies. Cancer and Met. Rev. 16:421-432, 1997.
19. Janssen, R. A., Mulder, N. H., The, T. H. and Leij, L. The immunobiological effects of interleukin-2 in vivo. Cancer Immunol. Immunother. 39:-207-216, 1994.
20. Leonard, J. P., Sherman, M. L., Fisher, G., Buchanan, L. J., Larsen, G., Atkins, M. B., Sosman, J. A., Dutcher, J. P., Vogelzang, N. J. and Ryan, J. L. Effects of single-dose Interleukin-12 exposure on interleukin-12-associated toxicity and interferon-□ production. Blood 90(7):2541-2548, 1997.
21. Gilboa, E. Immunotherapy of cancer with genetically modified tumor vaccines. Seminars in Oncol. 23(1):101-107, 1996.
22. Tuting, T., Storkus, W. J. and Lotze, M. T. Gene-based strategies for the immunotherapy of cancer. J. Mol. Med. 75:478-491, 1997.
23. Cavallo, F., Signorelli, P., Giovarelli, M., Musiani, P., Modesti, A., Brunda, M. J., Colombo, M. P. and Forni, G. Antitumor efficacy of adenocarcinoma cells engineered to produce interleukin 12 (IL-1 2) or other cytokines compared with exogenous IL-12. J. Natl. Cancer Inst. 89(14):1049-1058, 1997.
24. Sun, Y., Jurgovsky, K., Moller, P., Alijagic, S., Dorbic, T., Georgieva, J., Wittig, B. and Schadendorf, D. Vaccination with IL-12 gene-modified autologous melanoma cells: preclinical results and a first clinical phase I study. Gene Therapy 5:481-460, 1998.
25. Egilmez, N. K., Jong, Y. S., Sabel, M. S., Jacob, J. S., Mathiowitz, E. and Bankert, R. B. 2000. In situ tumor vaccination with Interleukin-12 encapsulated biodegradable microspheres: induction of tumor regression and potent antitumor immunity. Cancer Res. 60:3832.
26. Hill, H. C., Conway, T. F., Sabel, M. S., Jong Y. S., Mathiowitz, E., Bankert, R. B., Egilmez, N. K. Cancer Immunotherapy with Interleukin-12 and Granulocyte-Macrophage Colony-Stimulating Factorencapsulated microspheres: Coinduction of innate and adaptive immunity and cure of disseminated disease. Cancer Res. 62:7254-7263, 2002.
27. Rapaich Moser, A., Pitot, H. C. and Dove, W. F. A dominant mutation that predisposes to multiple intestinal Neoplasia in the mouse. Science 247:322-324, 1960.
28. Su L. K., Kinzler, K. W., Vogelstein, B., Preisinger, A. C., Rapaich Moser, A., Luongo, C., Gould, K. A. and Dove, W. F. Multiple intestinal neoplasia caused by a mutation in the murine homolog of the APC gene. Science 256:668-670, 1992.
29. Chiu, C. H., McEntee, M. F., Whelan, J. Sulindac causes reapid regression of preexisting tumors in Min/+ mice independent of prostaglandin biosynthesis. Cancer Res. 57(19):4267-73, 1997.
30. Egilmez, N. K., Jong, Y. S., Sabel, M. S., Jacob, J. S., Mathiowitz, E. and Bankert, R. B. In situ tumor vaccination with interleukin- 12 encapsulated biodegradable nanospheres: Induction of tumor regression and potent antitumor immunity. Cancer Res. 60:3832-3837, 2000.

1. A method of inhibiting the growth of gastrointestinal tumors comprising the steps of orally administering to an individual with one or more gastrointestinal tumors, a formulation comprising polymeric microspheres encapsulating a drug composition comprising an agent selected from the group consisting of sulindac, IL-12 or a combination thereof, wherein said oral administration of the encapsulated agent is effective in inhibiting the growth of the one or more gastrointestinal tumors.

2. The method of claim 1, wherein the polymer is a polyanhydride.

3. The method of claim 1, wherein the polyanhydride is selected from the group consisting of polylactic acid, polylactide-co-glycolide, polycaprolactone and poly(fumaric-co-sebacic anhydride).

4. The method of claim 1, wherein the polymeric microspheres are prepared by the phase inversion method.

5. The method of claim 1, wherein the polymeric microspheres are prepared by the hot melt method.

6. The method of claim 1, wherein the amount of sulindac administered is about 100-400 mg/dose.

7. The method of claim 1, wherein the amount of IL-12 administered is about 100-300 ng/kg.

8. The method of claim 1, wherein the gastrointestinal tumor is a colorectal tumor.

9. The method of claim 1, wherein the polymer of the polymeric microspheres is polylactic acid or poly(fumaric-co-sebacic acid) and the encapsulated agent is sulindac.

10. The method of claim 1, wherein the polymer of the polymeric microspheres is polylactic and the encapsulated agent is IL-12.

11. The method of claim 1, wherein the polymeric microspheres are administered to the individual in combination with a treatment selected from the group consisting of surgery, radiation, chemotherapy and immunotherapy.

12. A method of preventing the development of gastrointestinal tumors comprising the steps of orally administering to an individual a formulation comprising polymeric microspheres encapsulating a drug composition comprising an agent selected from the group consisting of sulindac, IL-12 or a combination thereof, wherein said oral administration of the formulation is effective in preventing the development of gastrointestinal tumors.

13. The method of claim 12, wherein the polymer of the polymeric microspheres comprises a polyanhydride.

14. The method of claim 13, wherein the polyanhydride is selected from the group consisting of polylactic acid, polylactide-co-glycolide, polycaprolactone and poly(fumaric-co-sebacic anhydride).

15. The method of claim 12, wherein the polymeric microspheres are prepared by the phase inversion method.

16. The method of claim 12, wherein the polymeric microspheres are prepared by the hot melt method.

17. The method of claim 12, wherein the amount of sulindac administered is about 100-400 mg/dose.

18. The method of claim 12, wherein the amount of IL-12 administered is about 100-300 ng/kg.

19. The method of claim 12, wherein the gastrointestinal tumor is a colorectal tumor.

20. The method of claim 12, wherein the polymer in the polymeric microspheres is polylactic acid or poly(fumaric-co-sebacic acid) and the encapsulated agent is sulindac.

21. The method of claim 12, wherein the polymer in the polymeric microspheres is polylactic acid and the encapsulated agent is IL-12.

22. The method of claim 12, the polymeric microspheres are administered in combination with a treatment selected from the group consisting of surgery, radiation, chemotherapy and immunotherapy.

23. A composition comprising polyanhydride microspheres, wherein the microspheres encapsulate an agent selected from the group consisting of sulindac, IL-12 or a combination thereof.

24. The composition of claim 23, wherein the polyanhydride is selected from the group consisting of polylactic acid, polylactide-co-glycolide, polycaprolactone and poly(fumaric-co-sebacic anhydride).

25. The composition of claim 24, wherein the polyanhydride is polylactic acid or poly (fumaric-co-sebacic acid) and the encapsulated agent is sulindac.

26. The composition of claim 24, wherein the polyanhydride is polylactic acid and the encapsulated agent is IL-12.
